(19) United States
(12) Patent Application Publication
(10) Pub. No.: US 2025/0185926 A1
(43) Pub. Date: Jun. 12, 2025

(54) MEASUREMENT DEVICE, CONTROL METHOD, CONTROL RECORDING MEDIUM

(71) Applicant: OMRON HEALTHCARE Co., Ltd., Kyoto (JP)

(72) Inventors: Yasunori FUKUDA, Kyoto (JP); Kazuki KUMAGAI, Kyoto (JP); Nozomi NAKAWAKI, Kyoto (JP); Yuya HIGUCHI, Kyoto (JP)

(21) Appl. No.: 19/057,590

(22) Filed: Feb. 19, 2025

Related U.S. Application Data
(63) Continuation of application No. PCT/JP2023/036917, filed on Oct. 11, 2023.

(30) Foreign Application Priority Data
Feb. 8, 2023 (JP) 2023-017653

(51) Int. Cl. A61B 5/021

(52) U.S. Cl. CPC A61B 5/02108; A61B 2560/045

(57) ABSTRACT

A measurement device, a control method, and a control recording medium that can reduce a processing load of a processor and suppress a delay in processing such as measurement are provided. A measurement device according to one aspect of the present invention includes a main MCU that performs measurement based on biological data obtained by a sensor, a communication IC that performs wireless communication with an information terminal, and a nonvolatile memory connected to the communication IC. The main MCU specifies an address of a write destination in the nonvolatile memory and causes the communication IC to write the biological data into the nonvolatile memory, and specifies an address of a read source in the nonvolatile memory and causes the communication IC to read the biological data from the nonvolatile memory and transmit the biological data to the information terminal.

### CROSS-REFERENCE TO RELATED APPLICATIONS

This application is the U.S. national stage application filed pursuant to 35 U.S.C. 365(c) and 120 as a continuation of International Patent Application No. PCT/JP2023/036917, filed Oct. 11, 2023, which application claims priority to Japanese Patent Application No. 2023-017653, filed Feb. 8, 2023, which applications are incorporated herein by reference in their entireties.

### TECHNICAL FIELD

The present invention relates to a measurement device, a control method, and a control recording medium.

### BACKGROUND ART

There is a known biological information measurement device including a communication unit for performing wireless communication with an external device such as a personal computer and capable of transmitting measured biological information to the external device (Patent Document 1). There is a known sphygmomanometer that can store a pulse wave detected by a blood pressure calculation means and a blood pressure value calculated from the pulse wave into a storage means in association with measurement date and time, output the stored data to the outside from an output terminal, and display a signal level of the pulse wave in a time-series graph (Patent Document 2).

### CITATION LIST

### Patent Literature

Patent Document 1: JP 2008-061663 A
Patent Document 2: JP 2007-098003 A

### SUMMARY

### Technical Problem

According to the biological information measurement device of Patent Document 1, it is possible to transmit measured biological information to an external device. According to the sphygmomanometer of Patent Document 2, it is possible to clearly indicate what pulse wave the blood pressure calculation means to detect and what feature point of the detected pulse wave the blood pressure calculation means to use to calculate the blood pressure value, and therefore it is possible for subjects themselves to confirm whether calculation of the blood pressure value is normally performed. However, Patent Document 1 and Patent Document 2 do not describe reducing a processing load of a processor that writes measured biological information into a memory or reads the biological information from the memory and transmits the biological information to an external device.

One aspect of the present invention has been made in view of such circumstances, and an object thereof is to provide a measurement device, a control method, and a control recording medium that can reduce a processing load of a processor and suppress a delay in processing such as measurement.

### Solution to Problem

The present invention employs the following configurations to solve the above-described problems.

(1)

A measurement device including:
a first processor that performs measurement based on biological data obtained by a sensor;
a second processor that performs wireless communication with an information terminal;
a memory connected to the second processor,
in which the first processor
specifies an address of a write destination in the memory and causes the second processor to write the biological data into the memory, and
specifies an address of a read source in the memory and causes the second processor to read the biological data from the memory and transmit the biological data to the information terminal.

According to (1), by providing the second processor that performs wireless communication separately from the first processor that performs measurement based on biological data, it is possible to distribute, to the second processor, a processing load for transmitting biological data such as pulse wave data to an information terminal, and reduce the processing load of the first processor. This can suppress a delay in processing such as measurement by the first processor. Since the first processor is configured to specify the address of the memory connected to the second processor and instruct the second processor to write, read, and transmit the biological data, flow control and delivery confirmation are unnecessary in an interface (universal asynchronous receiver transmitter (UART) or the like) between the first processor and the second processor, and the transfer speed of the biological data to the information terminal can be improved. Since the second processor may perform writing of information to a specified address in the memory and reading and transmission of information from the specified address in the memory, the second processor can have a simple configuration. By an instruction to the second processor, the first processor can flexibly perform writing of the biological data to the memory, reading of the biological data from the memory, and transmission of the read biological data. However, since it is not necessary for the first processor itself to perform processing with a large load such as write processing, read processing, and transmission processing of biological data and the like, it is possible to reduce the processing load of the first processor as described above. Note that the pulse wave may be a pressure pulse wave obtained by measuring a change in pressure applied to a blood vessel, or may be a volume pulse wave obtained by measuring a change in blood volume in the blood vessel.

(2)

The measurement device according to (1), in which
the memory has an area allocated for the biological data, and
the address of the write destination and the address of the read source are addresses in the area.

According to (2), by providing the memory with an area where information other than the biological data is not written, it is possible to suppress interference between writing of the biological data by an instruction from the first processor to the second processor and writing of other information by the second processor.

(3)

The measurement device according to (1) or (2), in which
the memory is inaccessible from the first processor.

According to (3), as compared with a configuration in which the first processor and the second processor share one memory, access processing can be distributed and sped up.

(4)

The measurement device according to any of (1) to (3), in which
the biological data is pulse wave data.

As in (4), as the biological data to be measured by the measurement device, for example, pulse wave data is preferable.

(5)

The measurement device according to (4), in which
the first processor outputs a blood pressure measurement result based on the pulse wave data.

According to (5), the first processor can perform wireless transmission of the pulse wave data to the information terminal and output of the blood pressure measurement result.

(6)

The measurement device according to any one of (1) to (5), in which the memory is a nonvolatile memory.

According to (6), large-volume biological data can be saved with an inexpensive configuration.

(7)

A control method of a measurement device including a first processor that performs measurement based on biological data obtained by a sensor, a second processor that performs wireless communication with an information terminal, and a memory connected to the second processor,
in which the first processor
specifies an address of a write destination in the memory and causes the second processor to write the biological data into the memory, and
specifies an address of a read source in the memory and causes the second processor to read the biological data from the memory and transmit the biological data to the information terminal.

According to (7), by providing the second processor that performs wireless communication separately from the first processor that performs measurement based on biological data, it is possible to distribute, to the second processor, a processing load for transmitting biological data such as pulse wave data to an information terminal, and reduce the processing load of the first processor. This can suppress a delay in processing such as measurement by the first processor. Since the first processor is configured to specify the address of the memory connected to the second processor and instruct the second processor to write, read, and transmit the biological data, flow control and delivery confirmation are unnecessary in an interface (UART or the like) between the first processor and the second processor, and the transfer speed of the biological data to the information terminal can be improved. Since the second processor may perform writing of information to a specified address in the memory and reading and transmission of information from the specified address in the memory, the second processor can have a simple configuration. By an instruction to the second processor, the first processor can flexibly perform writing of the biological data to the memory, reading of the biological data from the memory, and transmission of the read biological data. However, since it is not necessary for the first processor itself to perform processing with a large load such as write processing, read processing, and transmission processing of biological data and the like, it is possible to reduce the processing load of the first processor as described above.

(8)

A control recording medium of a measurement device including a first processor that performs measurement based on biological data obtained by a sensor, a second processor that performs wireless communication with an information terminal, and a memory connected to the second processor, the control recording medium for causing
the first processor to execute processing of
specifying an address of a write destination in the memory and causing the second processor to write the biological data into the memory, and
specifying an address of a read source in the memory and causing the second processor to read the biological data from the memory and transmit the biological data to the information terminal.

According to (8), by providing the second processor that performs wireless communication separately from the first processor that performs measurement based on biological data, it is possible to distribute, to the second processor, a processing load for transmitting biological data such as pulse wave data to an information terminal, and reduce the processing load of the first processor. This can suppress a delay in processing such as measurement by the first processor. Since the first processor is configured to specify the address of the memory connected to the second processor and instruct the second processor to write, read, and transmit the biological data, flow control and delivery confirmation are unnecessary in an interface (UART or the like) between the first processor and the second processor, and the transfer speed of the biological data to the information terminal can be improved. Since the second processor may perform writing of information to a specified address in the memory and reading and transmission of information from the specified address in the memory, the second processor can have a simple configuration. By an instruction to the second processor, the first processor can flexibly perform writing of the biological data to the memory, reading of the biological data from the memory, and transmission of the read biological data. However, since it is not necessary for the first processor itself to perform processing with a large load such as write processing, read processing, and transmission processing of biological data and the like, it is possible to reduce the processing load of the first processor as described above.

### Advantageous Effects of Invention

According to the present invention, it is possible to provide a measurement device, a control method, and a control recording medium that can reduce a processing load of a processor and suppress a delay in processing such as measurement.

### BRIEF DESCRIPTION OF DRAWINGS

FIG. 1 is a view illustrating an information management system including a measurement device of the present invention and an information terminal that performs wireless communication with the measurement device.

FIG. 2 is a view illustrating a sphygmomanometer that is an example of the measurement device.

FIG. 3 is a view illustrating an example in which the information terminal is connected to a network.

FIG. 4 is a block diagram illustrating a configuration of the measurement device.

FIG. 5 is a block diagram illustrating a configuration of the information terminal.

FIG. 6 is a sequence diagram illustrating operations of a main MCU, a communication IC, and a nonvolatile memory in the measurement device.

FIG. 7 is a view illustrating an example of biological data to be measured by the measurement device.

### DESCRIPTION OF EMBODIMENTS

An embodiment according to one aspect of the present invention will be described below based on the drawings.

### § 1 Application Example

<Information Management System 100 Applied with Present Invention>

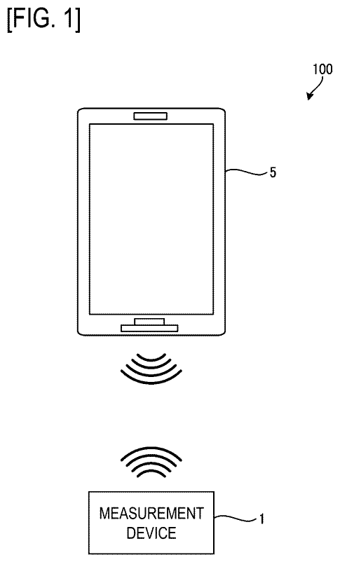

FIG. 1 illustrates an information management system 100 including a measurement device 1 of the present invention and an information terminal 5 that performs wireless communication with the measurement device 1.

The measurement device 1 includes a biological data measurement device that measures biological data such as a body weight, a body composition, blood pressure, a pulse, a heart rate, body temperature, blood glucose, or a blood oxygen saturation level. The measurement device 1 includes a measurement sensor for measuring a measurement target amount. The measurement target amount of the measurement sensor includes biological data such as a body weight, a body fat percentage, a blood pressure value, a pulse rate, a heart rate, body temperature, a blood glucose level, or a blood oxygen saturation level in accordance with the measurement device 1. The measurement device 1 is a non-wearable measurement device. The non-wearable measurement device is a measurement device that is not wearable. The wearable measurement device is a measurement device (e.g., an activity meter) carried by being worn on the body of a user. For example, the measurement device 1 (non-wearable measurement device) is a measurement device such as a scale, a body composition meter, a body weight-composition meter, or a sphygmomanometer that is used in a state of being installed on a ground or a table. The measurement device 1 transmits the measured biological data as measurement biological data of the user to the information terminal 5 by wireless communication.

The information terminal 5 stores the measurement biological data received from the measurement device 1 into a data storage unit in the information terminal 5. The information terminal 5 can perform wireless communication also with an external device other than the measurement device 1, and stores information acquired from the external device into the data storage unit in the information terminal 5. The information terminal 5 is an information processing device that analyzes various types of information acquired from the measurement device 1 and other external devices. The information terminal 5 is a terminal having a display such as, for example, a smartphone, a tablet terminal, a laptop computer, a desktop computer, or a wearable terminal. The information terminal 5 may be set so as to acquire measurement biological data from a specific measurement device 1. The specific measurement device 1 from which the measurement biological data is acquired may be registered in advance in the data storage unit of the information terminal 5.

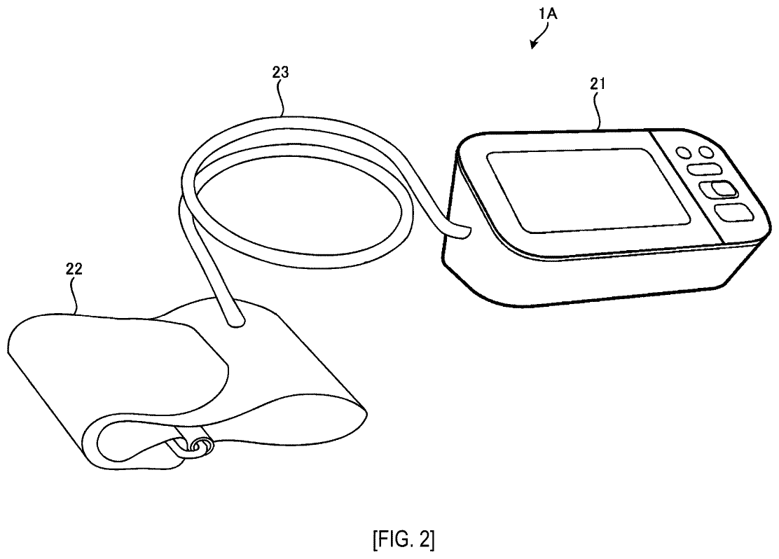

FIG. 2 is a view illustrating a sphygmomanometer 1A that is an example of the measurement device 1. The sphygmomanometer 1A is an example of a biological data measurement device, measures a blood pressure (pressure pulse wave data) of the user, and outputs a measurement result thereof to the user. The sphygmomanometer 1A transmits the measurement result as measurement biological data of the user to the information terminal 5 by wireless communication. For example, the sphygmomanometer 1A includes a main body portion 21, a cuff 22 that can be wound around an upper arm of the user, and an air tube 23 that connects the main body portion 21 and the cuff 22. In the example of FIG. 2, the cuff 22 and the main body portion 21 are separated, but the cuff 22 may be integrated with the main body portion 21.

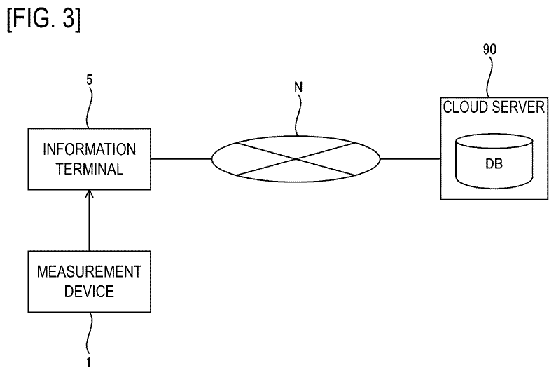

FIG. 3 is a view illustrating an example in which the information terminal 5 is connected to the network. As illustrated in FIG. 3, the information terminal 5 may be connected to a cloud server 90 via a wide area network N such as the Internet. The information terminal 5 may transmit the measurement biological data stored in the information terminal 5 to the cloud server 90 via the wide area network N, and the cloud server 90 may manage the measurement biological data of a user W as a database. The information terminal 5 may acquire, via the wide area network N, the measurement biological data managed by the cloud server 90 and use the acquired measurement biological data.

### § 2 Configuration Example

### <Configuration of Measurement Device 1>

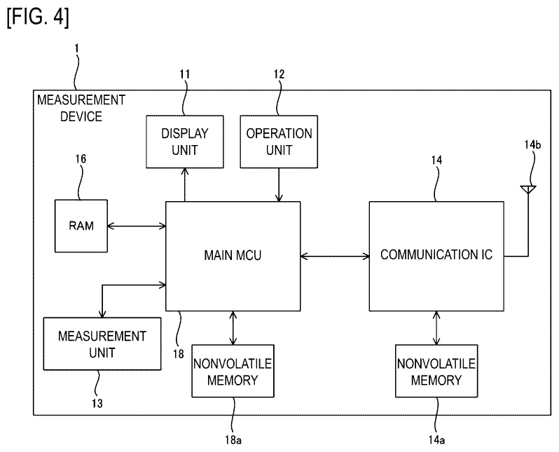

FIG. 4 is a block diagram illustrating the configuration of the measurement device 1. The measurement device 1 includes a display unit 11 that can display various types of information, an operation unit 12 that can be operated by the user, a measurement unit 13 that measures biological data and the like, a communication integrated circuit (IC) 14 that performs communication with an external device, a nonvolatile memory 14a connected to the communication IC 14, and a communication antenna 14b. The measurement device 1 includes a random access memory (RAM) 16 that temporarily stores information, a main micro controller unit (MCU) 18 that controls the operation of the entire device, and a nonvolatile memory 18a connected to the main MCU 18. The main MCU 18 is an example of the first processor of the present invention. The communication IC 14 is an example of the second processor of the present invention. As a communication interface between the main MCU 18 and the communication IC 14, for example, an interface such as a UART is used.

The display unit 11 includes a liquid crystal display or an organic electro luminescence (EL) display, for example. The operation unit 12 is a user interface that receives a user operation such as a button or a touch panel. The button includes a button physically provided at the measurement device 1 or a virtual button displayed on the display unit 11.

The measurement unit 13 includes a sensor that measures biological data such as a body weight, a body composition, blood pressure, a pulse, a heart rate, body temperature, blood glucose, and a blood oxygen saturation level. What to be measured varies depending on a measurement target of the measurement device 1.

The nonvolatile memory 14a is a recording medium that stores a parameter necessary for implementing a predetermined function, a control recording medium, and biological data measured by the measurement unit 13. The nonvolatile memory 14a includes, for example, a flash memory. The nonvolatile memory 14a is provided with a biological data area allocated for storing biological data. The biological data stored in the nonvolatile memory 14a is managed by the communication IC 14.

The communication IC 14 implements a predetermined function by executing a control recording medium. For example, the communication IC 14 can perform near-field wireless communication by executing a communication recording medium stored in the nonvolatile memory 14a. The communication IC 14 performs communication in compliance with, for example, Bluetooth Low Energy (BLE (registered trademark)) standard. The communication IC 14 transmits, in a cyclic period, an advertise signal for performing wireless communication to an unspecified number of external devices by broadcast communication. The communication IC 14 sends an advertise signal including, for example, the name and attribute information of the measurement device 1. The BLE communication performed by the communication IC 14 is, for example, communication using a 2.4 GHz frequency.

The communication IC 14 can manage the biological data by executing, for example, a management recording medium stored in the nonvolatile memory 14a. The biological data is biological data of the user measured by the measurement unit 13.

For example, the communication IC 14 performs write processing of writing the measured biological data into the nonvolatile memory 14a. The communication IC 14 performs read processing of reading the biological data from the nonvolatile memory 14a. The communication IC 14 writes the biological data into the nonvolatile memory 14a in accordance with a write instruction signal transmitted from the main MCU 18 to the communication IC, and reads the biological data from the nonvolatile memory 14a in accordance with a read instruction signal. The communication IC 14 performs write processing and read processing of the biological data with respect to the biological data area of the nonvolatile memory 14a. In the biological data area, information other than the biological data to be written in accordance with the write instruction signal from the main MCU 18 is not written. The biological data area is a dedicated area that can be used by the main MCU 18 in the area provided in the nonvolatile memory 14a.

The communication IC 14 performs transmission processing of transmitting the biological data read from the nonvolatile memory 14a to, for example, the information terminal 5 by wireless communication using the antenna 14b. The communication IC 14 performs transmission processing of the biological data in accordance with the transmission instruction signal transmitted from the main MCU 18 to the communication IC 14.

The RAM 16 includes a semiconductor device such as, for example, a dynamic RAM (DRAM) or a static RAM (SRAM), temporarily stores information, and also operates as a work area of the main MCU 18.

The nonvolatile memory 18a is a recording medium that stores a parameter necessary for implementing a predetermined function, a control recording medium, address information of a biological data area in the nonvolatile memory 14a connected to the communication IC 14, and the like. The nonvolatile memory 18a includes, for example, an electrically erasable programmable read only memory (EEPROM). Note that in the present example, the nonvolatile memory 14a has a configuration independent of the communication IC 14, but for example, the nonvolatile memory 14a may be one module with the communication IC 14.

The main MCU 18 implements a predetermined function by executing a control recording medium. For example, the main MCU 18 can perform measurement based on the biological data acquired by the measurement unit 13 by executing a measurement recording medium stored in the nonvolatile memory 18a.

The main MCU 18 can manage and instruct the measured biological data by executing, for example, a management instruction recording medium stored in the nonvolatile memory 18a. For example, the main MCU 18 transmits, to the communication IC 14, a write instruction signal for specifying an address of a write destination in the biological data area of the nonvolatile memory 14a and writing the biological data into the nonvolatile memory 14a. The main MCU 18 sequentially transmits the biological data obtained during the measurement by the measurement unit 13 to the communication IC 14 together with the write instruction signal. The main MCU 18 sequentially transmits the biological data to the communication IC 14 without receiving a response signal from the communication IC 14 to transmission of the biological data to the communication IC 14, that is, without performing delivery confirmation of the biological data. The sequential transmission of the biological data is to divide, at regular time intervals, and sequentially transmit the biological data, which is time-series data. When sequentially sending the biological data divided at regular time intervals, the main MCU 18 performs transmission (e.g., transmission by a streaming method) without performing delivery confirmation each time. The main MCU 18 attaches flag information indicating that the delivery confirmation as to whether or not the biological data has been delivered is not performed to the biological data and transmits the biological data to the communication IC 14. For example, the main MCU 18 transmits the biological data to the communication IC in 18 bytes every 32 msec.

The main MCU 18 transmits, to the communication IC 14, a read instruction signal for specifying an address of a read source in the biological data area of the nonvolatile memory 14a and reading the biological data from the nonvolatile memory 14a. Note that the address specification of write and read may be, for example, specification of a start address of write and read and specification of a size of write information or read information in the biological data area of the nonvolatile memory 14a, or specification of a start address and an end address of write and read.

The main MCU 18 can instruct the transmission processing of the communication IC 14 by executing, for example, a transmission instruction recording medium stored in the nonvolatile memory 18a. For example, the main MCU 18 transmits, to the communication IC 14, a transmission instruction signal for transmitting an advertise signal for wireless communication (BLE communication) in a cyclic period, and a transmission instruction signal for transmitting biological data read from the nonvolatile memory 14a to an external device such as the information terminal 5. When writing the measured biological data into the nonvolatile memory 14a, when reading the biological data, and when transmitting the biological data to the external device, the main MCU 18 only transmits, to the communication IC 14, an instruction signal including address specification of the nonvolatile memory 14a. Then, the write processing of the biological data to the nonvolatile memory 14a, the read processing of the biological data, and the transmission processing of the biological data to the external device are configured to be executed by the communication IC 14 having received an instruction from the main MCU 18. That is, the main MCU 18 is configured to be indirectly accessible to the nonvolatile memory 14a via the communication IC 14, but not directly accessible to the nonvolatile memory.

Before sensing of the biological data, the main MCU 18 transmits, to the communication IC 14, a saving start instruction signal that instructs starting of saving of the biological data into the nonvolatile memory 14a. After sensing of the biological data, the main MCU 18 transmits, to the communication IC 14, a saving end instruction signal that instructs ending of the saving of the biological data into the nonvolatile memory 14a. After the sensing of the biological data ends, the main MCU 18 receives, from the communication IC 14, result information regarding writing of the biological data into the nonvolatile memory 14a. The result information is transmitted by being included in a response signal from the communication IC 14 to the main MCU 18 in response to the instruction of ending the saving. The result information includes information indicating the number of receptions of the biological data that the communication IC 14 has received from the main MCU 18 and information indicating the number of failures of the biological data that the communication IC 14 has failed to write into the nonvolatile memory 14a. Note that the result information may be information indicating the number of successes of the biological data successfully written into the nonvolatile memory 14a.

By executing, for example, an information output recording medium stored in the nonvolatile memory 18a, the main MCU 18 outputs a biological measurement result based on measured biological data, for example, a blood pressure measurement result based on pressure pulse wave data. The main MCU 18 displays the blood pressure measurement result on a screen of the display unit 11 of the measurement device 1, for example. The main MCU 18 may output the blood pressure measurement result by voice from the measurement device 1 or may wirelessly transmit the blood pressure measurement result to the information terminal 5.

### <Configuration of Information Terminal 5>

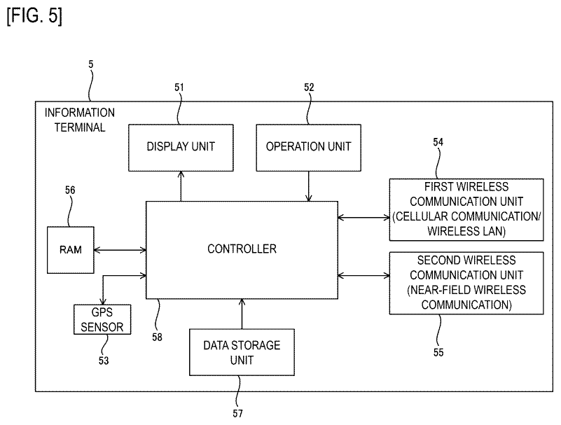

FIG. 5 is a block diagram illustrating the configuration of the information terminal 5. The information terminal 5 includes a display unit 51 that can display various types of information, an operation unit 52 than can be operated by the user, a global positioning system (GPS) sensor 53 for detecting a position, and a first wireless communication unit 54 and a second wireless communication unit 55 that perform communication with an external device. The information terminal 5 includes a RAM 56 that temporarily stores information, a data storage unit 57 that stores information and recording mediums, and a controller 58 that controls the operation of the entire terminal.

The display unit 51 includes a liquid crystal display or an organic electro luminescence (EL) display, for example. The operation unit 52 is a user interface that receives a user operation such as a button or a touch panel. The button includes a button physically provided at the information terminal 5 or a virtual button displayed on the display unit 51. The GPS sensor 53 is a sensor for detecting the current position of the information terminal 5.

The first wireless communication unit 54 is a communication unit that performs cellular communication, for example, a circuit (module) that can perform communication in compliance with a standard such as 4G, 5G, or long term evolution (LTE: registered trademark). The first wireless communication unit 54 is a communication unit that performs wireless LAN communication, for example, a circuit (module) that can perform communication in compliance with a standard such as Wi-Fi (registered trademark). The second wireless communication unit 55 is a communication unit that performs near-field wireless communication, for example, a circuit (module) for performing communication in compliance with the BLE standard.

The second wireless communication unit 55 acquires the biological data of the user measured by the measurement device 1, for example, by performing BLE communication with the communication IC 14 of the measurement device 1. The second wireless communication unit 55 receives the advertise signal transmitted from the communication IC 14 of the measurement device 1 by scanning. The second wireless communication unit 55 recognizes the measurement device 1 from the received advertise signal, and transmits a connection request to the measurement device 1 when communication connection is desired. Note that the measurement device 1 waits for a connection request for a predetermined time after transmitting the advertise signal, stops sending the advertise signal upon receiving the connection request within the predetermined time, and switches to one-to-one connection communication with a connection request partner.

The RAM 56 includes a semiconductor device such as a DRAM or an SRAM, for example, temporarily stores information, and operates as a work area of the controller 58.

The data storage unit 57 is a recording medium that stores a parameter necessary for implementing a predetermined function, a control recording medium, measurement biological data acquired from the measurement device 1, and the like. The data storage unit 57 includes, for example, a hard disk drive (HDD) or a semiconductor storage device (SSD).

The controller 58 implements a predetermined function by executing the control recording medium. Note that in the present embodiment, for example, management application software for an information terminal is installed in advance as a control recording medium in the data storage unit 57, and the controller 58 implements the predetermined function by executing this management application software. For example, when the management application software for the information terminal is started, the controller 58 controls the second wireless communication unit 55 so as to receive the advertise signal by scanning. When the advertise signal from the measurement device 1 is received, the controller 58 transmits a connection request to the measurement device 1 and controls the second wireless communication unit 55 so as to acquire the measurement biological data from the measurement device 1.

### § 3 Operation Example

### <Operation Example of Measurement Device 1>

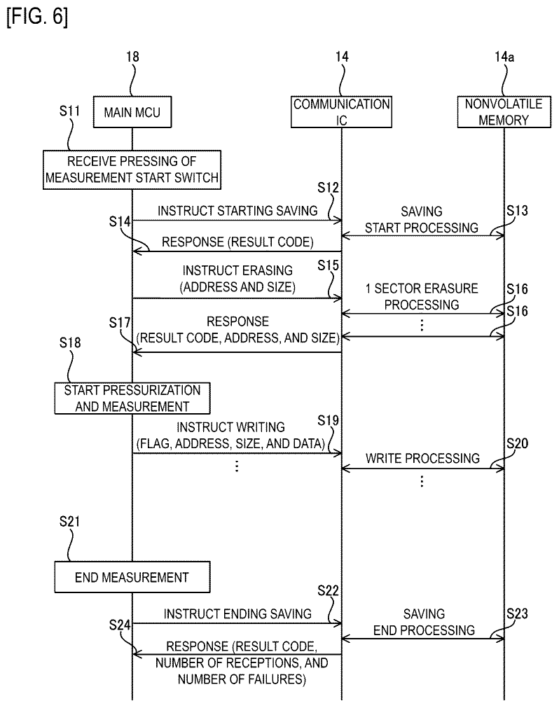

Next, an operation example of the measurement device 1 will be described with reference to FIG. 6. FIG. 6 is a sequence diagram illustrating operations of the main MCU 18, the communication IC 14, and the nonvolatile memory 14a in the measurement device 1. Note that the present example will be described below with the measurement device 1 being the sphygmomanometer 1A, and the biological information measured by the sphygmomanometer 1A being pressure pulse wave data.

It is assumed that an upper arm of the user is attached with the cuff 22 of the sphygmomanometer 1A and a measurement start switch is pressed.

First, the main MCU 18 receives pressing of the measurement start switch (step S11). Next, the main MCU 18 transmits, to the communication IC 14, a saving start instruction signal for instructing preparation for writing start (step S12).

Next, in response to the saving start instruction signal received in step S12, the communication IC 14 performs saving start processing for starting writing to the nonvolatile memory 14a (step S13). Next, the communication IC 14 transmits, to the main MCU 18, a response signal notifying that the saving start processing is completed (step S14). The response signal includes a result code indicating that the saving start processing is completed.

Next, upon receiving the response signal in step S14, the main MCU 18 transmits, to the communication IC 14, an erasure instruction signal for erasing the data in the nonvolatile memory 14a (step S15). The erasure instruction signal includes specification of an address of an area to be erased in the nonvolatile memory 14a and the size thereof.

Next, in response to the erasure instruction signal received in step S15, the communication IC 14 performs processing of erasing the data of the instructed area, for example, for each sector (step S16). The communication IC 14 repeats the erasure processing for 1 for each sector in accordance with the size of the instructed erasure area. The erasure processing for each sector is that, for example, when viewed from the communication IC 14 side, the communication IC 14 first transmits an erasure instruction for each sector to the nonvolatile memory 14a, and receives, from the nonvolatile memory 14a, a response indicative of having complied with the erasure instruction (a response indicating that one sector has been erased). Next, the communication IC 14 transmits, to the nonvolatile memory 14a, a read request for reading the erased one sector, receives, from the nonvolatile memory 14a, a response (read data of one sector) in response to the read request, then performs verification, and ends. Next, the communication IC 14 transmits, to the main MCU 18, a response signal notifying that the erasure processing is completed (step S17). The response signal includes a result code indicating that the erasure processing is completed, and an address and a data size of the erased data in the nonvolatile memory 14a.

Next, upon receiving the response signal in step S17, the main MCU 18 pressurizes the cuff 22 and starts measuring the pressure pulse wave data (step S18). Next, the main MCU 18 transmits, to the communication IC 14, a write instruction signal for writing the measured pressure pulse wave data into the biological data area of the nonvolatile memory 14a (step S19). The write instruction signal includes a flag indicating transmission (e.g., transmission by a streaming method) in which delivery confirmation as to whether or not the pressure pulse wave data has been delivered is not performed, an address of the nonvolatile memory 14a into which the pressure pulse wave data is written and a data size thereof, and the pressure pulse wave data to be written. The transmission of the pressure pulse wave data in this write instruction is sequentially performed by dividing, at regular time intervals, the pressure pulse wave data measured by the measurement unit 13.

Next, in response to the write instruction signal received in step S19, the communication IC 14 performs write processing of writing the pressure pulse wave data sequentially transmitted from the main MCU 18 into a specified address in the biological data area of the nonvolatile memory 14a for each pressure pulse wave data that is sequentially transmitted (step S20). The write processing for each pressure pulse wave data sequentially transmitted is that for example, when viewed from the communication IC 14 side, the communication IC 14 first transmits, to the nonvolatile memory 14a, a write instruction for each pressure pulse wave data sequentially transmitted from the main MCU 18, and receives, from the nonvolatile memory 14a, a response indicative of having complied with the write instruction (a response indicating that the pressure pulse wave data has been written). Next, the communication IC 14 transmits, to the nonvolatile memory 14a, a read request for reading the written pressure pulse wave data, receives, from the nonvolatile memory 14a, a response (read pressure pulse wave data) in response to the read request, then performs verification, and ends.

Next, upon ending the measurement of the pressure pulse wave data (step S21), the main MCU 18 transmits, to the communication IC 14, a saving end instruction signal instructing processing for ending writing (step S22).

Next, in response to the saving end instruction signal received in step S22, the communication IC 14 performs saving end processing for ending writing to the nonvolatile memory 14a (step S23). Next, the communication IC 14 transmits, to the main MCU 18, a response signal notifying that the saving end processing is completed (step S24). The response signal includes a result code indicating that the saving end processing is completed, the number of receptions of the pressure pulse wave data that the communication IC 14 has received from the main MCU 18, and the number of failures of the pressure pulse wave data having failed to be written to the nonvolatile memory 14a.

The transmission processing for transmitting the pressure pulse wave data written into the nonvolatile memory 14a from the sphygmomanometer 1A to the external information terminal 5 is performed, for example, after the measurement of the pressure pulse wave data described above ends. In this case, the main MCU 18 transmits, to the communication IC 14, a transmission instruction signal for transmitting the pressure pulse wave data. The transmission instruction signal includes specification of an address in the nonvolatile memory 14a of the pressure pulse wave data to be transmitted and the size thereof. Next, the communication IC 14 reads the pressure pulse wave data from the nonvolatile memory 14a in accordance with the transmission instruction signal received from the main MCU 18, and transmits the read pressure pulse wave data to the information terminal 5 by wireless communication. The communication IC 14 transmits, to the information terminal 5, result information (the number of receptions, the number of failures, and the like) regarding writing of the pressure pulse wave data together with the pressure pulse wave data.

### § 4 Measurement Data Example

### <Measurement Data of Measurement Device 1>

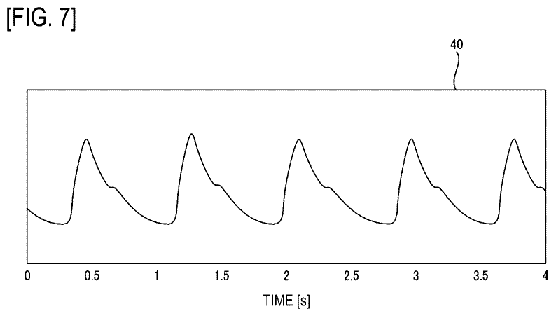

FIG. 7 is a view illustrating an example of biological data to be measured by the measurement device 1. The present example illustrates an example of pressure pulse wave data to be measured by the sphygmomanometer 1A. As illustrated in FIG. 7, pressure pulse wave data 40 is measured as a continuous conduction wave having substantially constant cyclicity. The pressure pulse wave data 40 having been measured is transmitted from the main MCU 18 to the communication IC. The main MCU 18 divides, at regular time intervals, and sequentially transmits, to the communication IC 14, the pressure pulse wave data 40 having been measured.

As described above, the main MCU 18 of the measurement device 1 specifies an address of a write destination in the biological data area of the nonvolatile memory 14a and causes the communication IC 14 to write the biological data into the nonvolatile memory 14a, and specifies an address of a read source in the biological data area of the nonvolatile memory 14a and causes the communication IC 14 to read the biological data from the nonvolatile memory 14a and transmit the biological data read from the nonvolatile memory 14a to an external device such as the information terminal 5 by wireless communication. According to this configuration, by providing the communication IC 14 that performs wireless communication separately from the main MCU 18 that performs measurement based on the biological data, it is possible to distribute, to the communication IC 14, the processing load for transmitting, to the information terminal 5, the biological data such as pressure pulse wave data, and reduce the processing load of the main MCU 18. This can suppress a delay in processing such as measurement by the main MCU 18. Since the main MCU 18 is configured to specify the address of the nonvolatile memory 14a connected to the communication IC 14 and instruct the communication IC 14 to write, read, and transmit the biological data, flow control and delivery confirmation are unnecessary in an interface between the main MCU 18 and the communication IC 14, and the transfer speed of the biological data to the information terminal 5 can be improved. Since the communication IC 14 may perform writing of biological data to a specified address in the nonvolatile memory 14a and reading and transmission of biological data from the specified address in the nonvolatile memory 14a, the communication IC 14 can have a simple configuration. By an instruction to the communication IC 14, the main MCU 18 can flexibly perform writing of the biological data to the nonvolatile memory 14a, reading of the biological data from the nonvolatile memory 14a, and transmission of the read biological data. However, since it is not necessary for the main MCU 18 itself to perform processing with a large load such as write processing, read processing, and transmission processing of biological data and the like, it is possible to reduce the processing load of the main MCU 18 as described above.

The measurement device 1 has a biological data area allocated for storing biological data in the nonvolatile memory 14a. The biological data stored in the biological data area is configured to be managed by the communication IC 14 and not accessible from the main MCU 18. According to this configuration, since information other than the biological data is not written into the biological data area, it is possible to suppress interference between writing of the biological data by an instruction from the main MCU 18 to the communication IC 14 and writing of other information by the communication IC 14. As compared with a configuration in which the main MCU 18 and the communication IC 14 share one nonvolatile memory, access processing can be distributed and sped up.

### § 5 Modified Examples

While an embodiment of the present invention has been described in detail above, the above description is merely illustrative of the present invention in all respects. Various modifications and variations can be made without departing from the scope of the present invention. For example, the following changes are possible. Note that in the following, the same reference signs are used for components that are the same as those of the above-described embodiment, and descriptions thereof are omitted as appropriate. The following modified examples can be combined as appropriate.

In the above embodiment, when the biological data measured by the main MCU 18 is written into the nonvolatile memory 14a, the biological data obtained during measurement by the measurement unit 13 is sequentially transmitted to the communication IC 14 without delivery confirmation and written, but no such limitation is intended. For example, the main MCU 18 may perform delivery confirmation of the biological data every time it sequentially transmits the biological data to the communication IC 14. In that case, since it is possible to obtain, by the delivery confirmation, the result information regarding the writing of the biological data to the nonvolatile memory 14a, it is not necessary to receive the result information from the communication IC 14 after the end of the sensing.

In the above embodiment, the configuration in which the biological data is pulse wave data and the pressure pulse wave data is acquired as pulse wave data has been described, but the measurement device 1 may be configured to measure volume pulse wave data as pulse wave data.

In the above embodiment, the nonvolatile memory 14a has been described as the memory connected to the communication IC 14 (second processor), but the memory connected to the communication IC 14 (second processor) is not limited to the nonvolatile memory 14a and may be a volatile memory or the like.

While various embodiments have been described above, needless to say, the present invention is not limited to such examples. It will be apparent to those skilled in the art that various changes and modifications can be made within the scope of the claims, and it should be understood that such changes and modifications also fall within the technical scope of the present invention. Components in the above-described embodiment may be combined as desired without departing from the gist of the invention.

Note that the present application is based on Japanese Patent Application (Japanese Patent Application No. 2023-017653) filed on Feb. 8, 2023, the contents of which are incorporated herein by reference.

### REFERENCE SIGNS LIST

1 Measurement device
1A Sphygmomanometer
5 Information terminal
11, 51 Display unit
12, 52 Operation unit
13 Measurement unit
14 Communication IC (second processor)
14a, 18a Nonvolatile memory
14b Antenna
16, 56 RAM
18 Main MCU (first processor)
21 Main body portion
22 Cuff
23 Air tube
40 Pressure pulse wave data
53 GPS sensor
54 First wireless communication unit
55 Second wireless communication unit
57 Data storage unit
58 Controller
90 Cloud server
100 Information management system

1. A measurement device comprising:
a first processor that performs measurement based on biological data obtained by a sensor;
a second processor that performs wireless communication with an information terminal;
a memory connected to the second processor and inaccessible from the first processor, wherein
the first processor
specifies an address of a write destination in the memory and causes the second processor to write the biological data into the memory, and
specifies an address of a read source in the memory and causes the second processor to read the biological data from the memory and transmit the biological data to the information terminal.

2. The measurement device according to claim 1, wherein
the memory has an area allocated for the biological data, and
the address of the write destination and the address of the read source are addresses in the area.

3. The measurement device according to claim 1, wherein
the biological data is pulse wave data.

4. The measurement device according to claim 3, wherein
the first processor outputs a blood pressure measurement result based on the pulse wave data.

5. The measurement device according to claim 1, wherein
the memory is a nonvolatile memory.

6. A control method of a measurement device including a first processor that performs measurement based on biological data obtained by a sensor, a second processor that performs wireless communication with an information terminal, and a memory connected to the second processor and inaccessible from the first processor, wherein
the first processor
specifies an address of a write destination in the memory and causes the second processor to write the biological data into the memory, and
specifies an address of a read source in the memory and causes the second processor to read the biological data from the memory and transmit the biological data to the information terminal.

7. A control recording medium of a measurement device including a first processor that performs measurement based on biological data obtained by a sensor, a second processor that performs wireless communication with an information terminal, and a memory connected to the second processor and inaccessible from the first processor, the control recording medium for causing
the first processor to execute processing of
specifying an address of a write destination in the memory and causing the second processor to write the biological data into the memory, and
specifying an address of a read source in the memory and causing the second processor to read the biological data from the memory and transmit the biological data to the information terminal.
